(19) United States
(12) Patent Application Publication
(10) Pub. No.: US 2002/0098558 A1
(43) Pub. Date: Jul. 25, 2002

(54) Method for producing L(+) -lactic acid using a specific enterococcus strain

(76) Inventors: Hwa-Won Ryu, Gwangju (KR); Jong-Sun Yun, Gwangju (KR)

Correspondence Address: Anderson Kill & Olick, P.C., 1251 Avenue of the Americas, New York, NY, 10020-1182, US

(21) Appl. No.: 10/020,542

(22) Filed: Oct. 29, 2001

(30) Foreign Application Priority Data
Nov. 29, 2000 (KR) 2000-71730

(51) Int. Cl. C12P 7/56

(52) U.S. Cl. 435/139

(57) ABSTRACT

An L(+)-lactic acid can be produced in a high yield by the steps of (a) pre-culturing Enterococcus sp. RKY1 KCTC 10092BP successively in a lactic acid-containing culture medium and in a glucose-containing culture medium; and (b) cultivating the pre-cultured Enterococcus sp. RKY1 KCTC 10092BP in a culture medium containing a carbon source, e.g., glucose, fructose and maltose.

### FIELD OF THE INVENTION

The present invention relates to a high-yield method for producing L(+)-lactic acid from such an inexpensive carbon source as monosaccharide and disaccharide using Enterococcus sp. RKY1.

### DESCRIPTION OF THE PRIOR ART

Lactic acid is a commercially valuable organic acid used in various fields including food processing, medicine, brewing, tanning, dying and optical materials, and it is also used as a starting material for producing a polylactide, an environmentally friendly commodity plastic that has high mechanical strength, thermal plasticity, fabric ability, biodegradability, and biocompatibility for uses in service ware, grocery, waste and composting bags, mulch films, and controlled release matrices of fertilizer, pesticides, and herbicides.

Furthermore, since lactic acid has an excellent reactivity that stems from it having both a carboxylic acid and hydroxyl group, it can undergo a variety of chemical conversions into potentially useful alternative chemicals such as propylene oxide, propylene glycol, acrylic acid, 2,3-pentanedione, and lactate ester

Lactic acid is produced both by fermentation and by chemical synthesis. Unlike the chemical methods which provide racemic lactic acid, either D(−)- or L(+)-lactic acid can be selectively produced by fermenting a carbohydrate as a carbon source, depending on the kind of microorganism used.

Fermentative processes for producing lactic acid have usually been conducted using such microorganisms as Lactobacillus and Lactococcus. For example, Korean Patent No. 122436 discloses a method for producing D(−)-or L(+)-lactic acid by using Lactobacillus casei subspecies rhamnosus, but it requires a long production time (48 hours) and gives a low productivity (2.5 g/L/hr).

The use of other microorganisms has also been attempted in the fermentative production of lactic acid. Korean Patent No. 122428 discloses a method of producing D(−)-lactic acid using an E. coli strain which lacks acetic acid production capability. However, this method requires a long fermentation time, e.g., 57 hours, and gives both a low concentration of lactic acid (60 g/L) and a low production rate (1.1 g/L/hr).

One prior art reference reports on lactic acid production using Enterococcus (Giurka et al., Journal of Food Biochemistry, 1993, 16 pp277-289), but it also shows a low yield (45%), a low lactic acid purity, and a low production rate (0.1 g/L/hr).

The present inventors have disclosed a method for the preparation of succinic acid which comprises culturing Enterococcus sp. RKY1 in a culture medium containing a glycerol or glucose as an electron donor and fumaric acid as an electron acceptor, and bioconverting fumaric acid into succinic acid using the cultured Enterococcus sp. RKY1(Korean Patent Publication No. 2000-9147), said Enterococcus sp. RKY1 being deposited on Jun. 1, 1998 with the Korean Collection for Type Cultures (KCTC)(Address: Korea Research Institute of Bioscience and Biotechnology (KRIBB), #52, Oun-dong, Yusong-ku, Taejon, 305-333, Republic of Korea) and the original deposit was converted to a deposit under the accession number, KCTC 10092BP, in accordance with the terms of Budapest Treaty on the International Recognition of the Deposit of Microorganism for the Purpose of Patent Procedure.

The present inventors have unexpectedly found that an acid- and sugar-tolerant strain of Enterococcus sp. RKY1 is exceptionally effective in the fermentative production of lactic acid.

### SUMMARY OF THE INVENTION

Accordingly, it is a primary object of the present invention to provide a method for producing lactic acid, particularly L(+)-lactic acid, in a high yield and at a high production rate using an acid- and sugar-tolerant strain of Enterococcus sp. RKY1.

In accordance with one aspect of the present invention, there is provided a method for producing lactic acid comprising (a) pre-culturing Enterococcus sp. RKY1 KCTC 10092BP in a lactic acid-containing culture medium and also in a glucose-containing culture medium in order to confer thereon acid-tolerance and sugar-tolerance, respectively; and (b) incubating a culture medium containing the acid- and sugar-tolerant Enterococcus sp. RKY1 KCTC 10092BP and a carbohydrate.

### DETAILED DESCRIPTION OF THE INVENTION

The present invention relates to the preparation of lactic acid, particularly L(+)-lactic acid, using Enterococcus sp. RKY1 KCTC 10092BP which is capable of converting a carbon source to lactic acid. Enterococcus sp. RKY1 KCTC 10092BP can grow in an aerobic or anaerobic condition, and does not require highly pure CO₂ in such a fermentation process.

Enterococcus sp. RKY1 KCTC 10092BP which can be used in the present invention is preferably subjected to two pre-culturing steps, which are each carried out at 20 to 50° C., preferably 38° C., for 12 to 24 hours.

The pre-culturing in a lactic acid-containing culture medium is carried out so that Enterococcus sp. RKY1 KCTC 10092BP can attain acid-tolerance, wherein the concentration of lactic acid may be 10 to 90 g/L based on the amount of the culture medium. Preferably, the lactic acid content of the medium is gradually increased within the range with time. The second pre-culturing step is carried out in a glucose- and lactic acid-containing culture medium for the microorganism to attain sugar- and acid-tolerance. The concentrations of glucose and lactic acid in the medium may be 10 to 150 g/L and 20 to 60 g/L, respectively, based on the amount of the culture medium. The glucose and lactic acid contents of the medium are preferably increased within said ranges with culture time.

The acid- and sugar-tolerant Enterococcus sp. RKY1 KCTC 10092BP thus obtained may then be used in fermenting a culture medium containing a carbon source to produce lactic acid, wherein the amount of said Enterococcus sp. RKY1 KCTC 10092BP is 2.0 to 10.0% of the culture medium, preferably 4.0% and the fermentation process may be conducted at 20 to 50° C., preferably 38° C., for 20 to 55 hours.

The carbon source which can be used in the present invention is selected from the group consisting of glucose, fructose, maltose and a mixture thereof, and it is used in an amount of 100 to 250 g/L, preferably 150 g/L based on the amount of the culture medium.

The culture medium may further comprise a nitrogen source and trace amounts of other substances which are commonly used in the cultivation of microorganisms. The nitrogen source includes a yeast extract, peptone, soytone, tryptone, ammonium sulfate, a maltose extract, and a mixture thereof. The other substances may be sodium chloride and potassium phosphate.

According to the present invention, lactic acid can be obtained from an inexpensive carbohydrate within a short reaction time and in a high yield, with a high productivity of 5.3 g/L/hr. Further, the isomer purity of lactic acid produced from carbon source, i.e., the weight of L(+)-lactic/total weight of lactic acid (g/g) is over 99%.

While the invention has been described with respect to the specific embodiments, it should be recognized that various modifications and changes may be made by those skilled in the art to the invention which also fall within the scope of the invention as defined by the appended claims.

### EXAMPLE

Preparation 1: Acid- and Sugar-tolerant Enterococcus sp. RKY1 KCTC 10092BP

Enterococcus sp. RKY1 KCTC 10092BP was cultured in a basal culture medium containing lactic acid, glucose (20 g/L), yeast extract (10 g/L), potassium phosphate (10 g/L) and sodium chloride (1 g/L) in a 20 ml-serum bottle, at 38° C. for 24 hours, while increasing the lactic acid content sequentially from 20 to 40 to 60 g/L.

The culture broth obtained above was then transferred to a box of culture medium containing glucose, and lactic acid (60 g/L) and cultured at 38° C. for 24 hours while raising the glucose content sequentially from 40 to 80 to 120 g/L, to obtain a sugar- and acid-tolerant Enterococcus sp. RKY1 KCTC 10092BP.

Preparation 2: Pre-cultivation of Enterococcus sp. RKY1 KCTC 10092BP

0.5 ml of a glycerol suspension of Enterococcus sp. RKY1 KCTC 10092BP under freeze-storage was inoculated to 14 ml of a culture medium containing lactic acid (60 g/L), glucose (120 g/L), yeast extract (10 g/L), potassium phosphate (10 g/L) and sodium chloride (1 g/L) in a 20 ml of serum bottle, and the medium was cultured in a shaking incubator operated at 200 rpm at 38° C. for 12 hours.

The resulting broth (3 ml) was transferred to 37 ml of a culture medium containing glucose (30 g/L), yeast extract (10 g/L), potassium phosphate (10 g/L) and sodium chloride (1 g/L) in a 50 ml of serum bottle, followed by culturing in a shaking incubator operated at 200 rpm at 38° C. for 6 hours, to obtain a modified Enterococcus sp. RKY1 KCTC 10092BP having both acid-tolerance and sugar-tolerance.

### Example 1

### Preparation of Lactic Acid

960 ml of a sterilized culture medium containing glucose, yeast extract, potassium phosphate and sodium chloride at concentrations of 150 g/L, 20 g/L, 10 g/L and 1 g/L, respectively, was placed in a 2.5 L fermentator (KF-2.5 L, Korea Fermentor Co., Republic of Korea) and was inoculated with 40 ml of the modified Enterococcus sp. RKY1 KCTC 10092BP obtained in Preparation 2. The resulting medium was incubated at 38° C. with stirring at 200 rpm, while the pH was continuously adjusted to 7 using 6N-Na₂CO₃.

The cell concentration was determined by measuring the absorbance at 660 nm with a spectrophotometer (UV-106A, Shimadzu Co., Japan) and the concentration of lactic acid produced was analyzed with HPLC (Waters Ltd., USA) with HPX-87H column (Bio-Rad Co., USA). L(+)-Lactic acid was quantified by an enzymatic methodusing the diagnostic kit (826-B) supplied by Sigma Co.(USA), and D(−)-lactic acid was determined with the diagnostic kit modified by replacing L(+)-lactic dehydrogenase with D(−)-lactic dehydrogenase (L-2395, Sigma Co., USA). The result is shown in Table 1.

### Example 2

The procedure of Example 1 was repeated except that fructose was used in place of glucose. The result is shown in Table 1.

### Example 3

The procedure of Example 1 was repeated except that maltose was used in place of glucose. The result is shown in Table 1.

TABLE 1

| | Example | | |
|---|---|---|---|
| | 1 | 2 | 3 |
| Carbon source | Glucose | Fructose | Maltose |
| Culture time (hrs) | 27 | 24 | 27 |
| Lactic acid (g/L) | 144.3 | 144.1 | 143.2 |
| L(+)-lactic acid (g/L) | 143.0 | 143.2 | 142.2 |
| D(−)-lactic acid (g/L) | 1.3 | 0.9 | 1.0 |
| Isomer purity (%)* | 99.1 | 99.4 | 99.3 |
| Dry cell weight (g/L) | 18.7 | 19.8 | 18.4 |
| Yield (%) | 96.2 | 96.1 | 95.5 |
| Productivity (g/L/hr) | 5.34 | 6.00 | 5.30 |

*isomer purity: g-L(+)-lactic acid/g-total lactic acid

### Example 4-7

The procedure of Example 1 was repeated except that the amount of yeast extract was varied as in Table 2. The result is shown in Table 2.

TABLE 2

Example

| | 4 | 5 | 6 | 7 |
|---|---|---|---|---|
| Amount of yeast | 10 | 15 | 20 | 30 |
| extract (g/L) | | | | |
| Culture time (hrs) | 48 | 39 | 27 | 24 |
| Lactic acid (g/L) | 135.8 | 139.6 | 142.5 | 143.9 |
| Dry Cell weight (g/L) | 7.1 | 9.5 | 14.2 | 17.3 |
| Yield (%) | 90.5 | 93.1 | 95.0 | 95.9 |
| Productivity (g/L/hr) | 2.83 | 3.58 | 5.28 | 6.00 |

### Example 8-11

The procedure of Example 1 was repeated except that the carbon source was varied as in Table 3. The result is shown in Table 3.

TABLE 3

| | Example | | | |
|---|---|---|---|---|
| | 8 | 9 | 10 | 11 |
| Carbon source | Glucose 75 | Fructose 75 | Glucose 125 | Glucose 25 |
| (g/L) | Fructose 75 | Maltose 75 | Maltose 25 | Maltose 125 |
| Culture Time | 27 | 39 | 35 | 55 |
| (hrs) | | | | |
| Lactic acid | 145.5 | 111.7 | 139.5 | 139.2 |
| (g/L) | | | | |
| Dry cell | 15.8 | 13.4 | 16.5 | 15.3 |
| weight (g/L) | | | | |
| Productivity | 5.38 | 2.86 | 3.99 | 2.53 |
| (g/L/hr) | | | | |

The results shown in Table 1, 2 and 3 demonstrate that such carbon sources as glucose, fructose, maltose and a mixture thereof can be converted to lactic acid, particularly L(+)-lactic acid in a high enantiomeric purity (g-L(+)-lactic acid/g-total lactic acid) of over 99% and in a high yield (g-total lactic acid/g-carbon source) of over 95%, within a relatively short culture time.

While the invention has been described with respect to the specific embodiments, it should be recognized that various modifications and changes may be made by those skilled in the art to the invention which also fall within the scope of the invention as defined as the appended claims.

What is claimed is:

1. A method for producing L(+)-lactic acid comprising cultivating a modified strain of Enterococcus sp. RKY1 KCTC 10092BP having both acid- and sugar-tolerance, in a culture medium containing a carbon source.

2. The method of claim 1, wherein the modified strain is obtained by subjecting Enterococcus sp. RKY1 KCTC 10092BP to two pre-culturing steps, one in a first culture medium containing lactic acid and the other, in a second culture medium containing both glucose and lactic acid.

3. The method of claim 2, wherein the lactic acid concentration of the first culture medium is 10 to 90 g/L, and the concentrations of glucose and lactic acid of the second culture medium are 10 to 150 g/L and 20 to 60 g/L, respectively, based on the amount of the culture medium.

4. The method of claim 3, wherein the concentration of lactic acid of the first medium and the concentration of the second medium are each increased within said ranges during culturing.

5. The method of claim 3 or 4, wherein the pre-culturing steps are each carried out at 20 to 50° C. for 12 to 24 hours.

6. The method of claim 1, wherein the carbon source is selected from the group consisting of glucose, fructose, maltose, and a mixture thereof.

7. The method of claim 6, wherein the concentration of the carbon source is 100 to 250 g/L based on the amount of the culture medium.

8. The method of claim 1, wherein the cultivating is carried out at 20 to 50° C. for 20 to 55 hours.
